(19) United States
(12) Patent Application Publication
(10) Pub. No.: US 2021/0090097 A1
(43) Pub. Date: Mar. 25, 2021

(54) COMPUTER SYSTEM AND METHOD FOR MARKET RESEARCH USING AUTOMATION AND VIRTUALIZATION

(71) Applicant: Delvinia Holdings Inc., Toronto (CA)

(72) Inventors: Adam Froman, Toronto (CA); Steve Mast, Toronto (CA)

(21) Appl. No.: 17/029,449

(22) Filed: Sep. 23, 2020

Related U.S. Application Data
(60) Provisional application No. 62/903,967, filed on Sep. 23, 2019.

(51) Int. Cl. G06Q 30/02; G06Q 10/10; G06Q 10/06; G06F 9/54; G06N 20/00; H04L 12/58; H04L 29/06; G06F 40/186; A61B 5/16; A61B 5/04

(52) U.S. Cl. CPC G06Q 30/0201; G06Q 10/10; G06Q 30/0203; G06Q 30/0204; G06Q 10/067; G06F 9/547; A61B 2503/12; H04L 51/02; H04L 67/42; G06F 40/186; A61B 5/163; A61B 5/165; A61B 5/04001; G06N 20/00

(57) ABSTRACT

Computer systems, methods, and devices for automated market research are provided. The system includes a research automation server platform. The research automation platform is configured to: store data collection method template data; generate an electronic data collection method using the data collection method template data and researcher input data; receive response data via an input interface based on a respondent interaction with the electronic data collection method; generate market research insight data; and display the market research insight data via an output interface. The research automation platform includes a client layer software component, a services layer software component, and a technology layer software component. The client layer software component and the services layer software component communicate via a client-services API layer. The services layer software component and the technology layer software component communicate via a services-technology API layer.

### TECHNICAL FIELD

The following relates generally to market research, and more particularly to systems and methods for automated market research.

### INTRODUCTION

Automated market research and consumer insights gathering is a fast-growing field. Brands and marketers are looking to include customer insights earlier and more often in their research and marketing processes. This may include quick tests throughout the process that may increase the chances of launching a successful project.

To meet these demands, brands and companies are seeking deeper customization of automated research platforms, including greater flexibility and customization of research methodologies or “methods”. Researchers are looking to collect data and gain insights faster, cheaper, and more effectively. Researchers want faster results from various parts of the research process from data collection to key insight reporting. There is a demand among researchers for more sophisticated and automated research reporting and analysis tools.

Traditionally, market research testing such as concept testing, advertising testing, pre-concept work, idea sorting, package design testing, evaluation and in-market testing for things like recall has been conducted via long, complex research studies that can be slow and costly. As capturing data and receiving insights and results faster becomes increasingly a priority, marketing executives are searching for new methods and channels to access consumer opinions. Many of these new channels, however, lack the rigor of traditional methodologies. Newer research approaches that include testing and analysis that provides instant feedback using AI-trained technologies may be difficult to integrate and challenging to work with for less technically savvy researchers (e.g. non-developers) and have yet to be widely adopted by researchers.

The current market research technology marketplace has grown to thousands of options for market researcher users to consider. Such technology options may be useful to researchers, but increasingly researchers do not have the time (or sometimes the capacity) to source, vet, or integrate applications into their research stacks.

Further, business decision makers influencing the market research process such as marketing professionals, business executives, and product managers are often non-developers. These non-developer researchers may not have the skills and expertise to work with and gain useful insights from existing market research technologies that may seem complicated and complex. This lack of expertise and ease-of-use may prevent adoption of certain technologies in their research processes or may require the expertise of additional personnel having developer experience, which can further complicate the process, reduce speed, and increase costs.

In some cases, market research technologies and applications cannot be easily engaged by non-developer researchers. For example, the technology may not be readily accessible to non-developers or the non-developer must manage using different user interfaces for each market research technology.

Existing approaches to providing market research and consumer insight tools often include a core technology, often machine learning or artificial intelligence-based, that may be augmented or integrated with software tailoring the technology to a specific use case in the market research space. The development of the core technology and application to the market research space are often done by different entities. As a result, the adoption of such technologies by researchers in their research process, including the ability to integrate results, can be complex and complicated.

These challenges with existing approaches to the automation of market research technology limit the effective adoption of new and useful market research technologies. This may, in turn, limit the speed and effectiveness of market research efforts.

There is a need for an online research platform that can provide a similar level of robustness as compared to custom data collection methods such as researcher-led or research consultant-led data collection methods, while also providing ease-of-use. Such a platform may increase the speed at which marketing assets can be tested and analyzed, and at which insights can be reported (e.g. reduce research testing time that may normally take two weeks to as little as hours).

Accordingly, there is a need for improved systems and methods for automated market research that overcome at least some of the disadvantages of existing approaches.

### SUMMARY

Systems, methods, and devices for automated market research are provided. The system includes a research automation server platform. The research automation platform is configured to: store data collection method template data; generate an electronic data collection method using the data collection method template data and researcher input data; receive response data via an input interface based on a respondent interaction with the electronic data collection method; generate market research insight data based on the response data; and display the market research insight data via an output interface. The research automation platform includes a client layer software component, a services layer software component, and a technology layer software component. The client layer software component and the services layer software component communicate via a client-services application programming interface (“API”) layer. The services layer software component and the technology layer software component communicate via a services-technology API layer.

The client layer software component may be configured to automate or virtualize a process performed by the research automation server platform.

The response data may include a survey participant response to market research data presented via the data collection method.

The market research data may include an image, a video, or an interactive prototype.

The researcher input data may include method selection data, audience selection data, and market research data.

The research automation server platform may be further configured to generate an audience of respondents for the electronic data collection method using the researcher input data.

The audience may be a virtual audience comprising a simulated model of a customer segment.

The electronic data collection method may be configured to test against the simulated model of the customer segment in real-time.

The research automation server platform may be further configured to define an audience for the electronic data collection in response to input data provided by a user. The audience may be an existing audience stored by the research automation server platform and previously created by the user using the research automation server platform.

The research automation server platform may be further configured to define an audience for the electronic data collection method using audience configuration data provided by a user and store the audience configuration data such that the audience configuration data can be used to define an audience for a subsequent electronic data collection method.

The response data may be provided by a virtual participant.

The virtual participant may represent a plurality of participants.

The research automation platform may include a method selection module for selecting the electronic data collection method from a plurality of data collection methods.

The method selection module may provide research needs data to a virtual recommendation engine in communication with the method selection module and the virtual recommendation engine may generate method suggestion data including at least one of the plurality of data collection methods.

The research automation server platform may include a virtualization module which implements a virtual researcher configured to perform any one or more of recommending a data collection method, identifying insights from the response data, and validating a concept tested using the electronic data collection method.

The electronic data collection method may include a chatbot configured to conduct an interview with a respondent.

The electronic data collection method may collect response data using any one or more of facial coding, emotion detection, eye-tracking, biometrics, or neuro sensors.

The market research insight data may be continually updated as new response data is received.

The response data may be generated by a machine learning model configured to simulate a human response to a particular question or stimuli.

The market research insight data may be generated using emotional analysis. The emotional analysis may be performed using an artificial intelligence system configured to simulate a human emotional reaction to stimuli presented in the electronic data collection method.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a schematic diagram of a system for automated market research, according to an embodiment;

FIG. 2 is a block diagram of a computing device of FIG. 1;

FIG. 3 is a block diagram of a layered market research system, according to an embodiment;

FIG. 4 is a block diagram of a software framework for an automated market research system, according to an embodiment;

FIG. 5 is a block diagram of software components of the market research server of FIG. 1, according to an embodiment;

FIG. 6 is a flow diagram of various modules in a research automation platform, according to an embodiment;

FIG. 7 is a flow diagram of a basic configuration module of a research automation platform, according to an embodiment;

FIG. 8 is a flow diagram of an audience configuration module of a research automation platform, according to an embodiment;

FIG. 9 is a flow diagram of a survey configuration module of a research automation platform, according to an embodiment;

FIG. 10 is a flow diagram of a data collection method configuration module of a research automation platform, according to an embodiment;

FIG. 11 is a flow diagram of a deployment and user review module of a research automation platform, according to an embodiment; and

FIG. 12 is a flow diagram of a going live module of a research automation platform, according to an embodiment.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

One or more systems described herein may be implemented in computer programs executing on programmable computers, each comprising at least one processor, a data storage system (including volatile and non-volatile memory and/or storage elements), at least one input device, and at least one output device. For example, and without limitation, the programmable computer may be a programmable logic unit, a mainframe computer, server, and personal computer, cloud-based program or system, laptop, personal data assistance, cellular telephone, smartphone, or tablet device.

Each program is preferably implemented in a high-level procedural or object-oriented programming and/or scripting language to communicate with a computer system. However, the programs can be implemented in assembly or machine language, if desired. In any case, the language may be a compiled or interpreted language. Each such computer program is preferably stored on a storage media or a device readable by a general or special purpose programmable computer for configuring and operating the computer when the storage media or device is read by the computer to perform the procedures described herein.

A description of an embodiment with several components in communication with each other does not imply that all such components are required. On the contrary, a variety of optional components are described to illustrate the wide variety of possible embodiments of the present invention.

Further, although process steps, method steps, algorithms or the like may be described (in the disclosure and/or in the claims) in a sequential order, such processes, methods and algorithms may be configured to work in alternate orders. In other words, any sequence or order of steps that may be described does not necessarily indicate a requirement that the steps be performed in that order. The steps of processes described herein may be performed in any order that is practical. Further, some steps may be performed simultaneously.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The following relates generally to systems and methods for market research, and more particularly to systems and methods for market research using automation and virtualization.

In an embodiment, a market research system is provided. The market research system includes a plurality of software layers. The software layers include a client layer, a services layer, and a technology layer. Software layers may communicate with one another via APIs. In a particular case, the market research system automates and/or virtualizes processes and functions which may otherwise be performed by a human researcher (of which there are numerous) or other individual (e.g. project managers, survey programmers, sample specialists, etc.). In doing so, the market research system may become more efficient. The service layer is “compressed” by a technology, which may include automation enabling less human involvement in the service layer, thereby “compressing” it.

In some cases, the market research system may compress the service layer by making service layer applications or software modules (or portions thereof) accessible to the client layer via an API. The API may be configured to coordinate requests and responses between software components in different layers including specifying specific data to be accessed and transferred to perform various automation and virtualization functions of the market research system. In doing so, the market research may be more efficient, limiting transferred data to data necessary to perform the function. For example, the market research system may take only the data needed by the user (as dictated by the user) to perform the task or function.

The market research system may include a common application that provides a consistent interface for accessing software components in the services and technology layers. This may increase automation and virtualization of the market research system, which may have advantages such as increasing speed and improving insight gathering.

The market research system may allow a user, such as a non-developer market researcher, to access and use a market research platform having a consistent user experience and interface that also is able to integrate and utilize various market research software components through a uniform interface. The market research software components may have application beyond market research but have been adapted, for example via the service layer, to provide functionalities tailored or directed to one or more market research tasks. The system may provide a single market research system platform through which a user can access and use various software applications and technologies for market research tasks to gain insights.

The market research system may provide faster on-boarding of new customers. Onboarding involves getting a customer or end-user of the market research system up and running on the system. This may involve customizing parts of the market research platform such as methodologies, reporting, and features or added services. The customer may be a “researcher” or “insights professional” who has an account login. This may be one or multiple people in an organization's single account.

The market research system may enable quicker customization of data collection methods and integration of research technologies.

Customization of a data collection method may include any one or more of changing or adding questions, defining a different audience besides a general population, adding reporting features, and changing how the algorithms draw and generate insights from the data.

A data collection method (also referred to herein as “method”) is the way that data is collected by the market research system and how insights are drawn from the collected data. A method may include an audience, a survey, and reporting. Some methods may have add-ons such as video response.

The data collection method may be a survey. The method may be a chatbot that conducts an interview with a participant. The data collected via the data collection method may include text, audio, video, or images. The method may include data collected via facial coding (emotion detection) or eye-tracking, biometrics or neuro sensors. Customizing a data collection method may include manually changing elements of the method. For example, the system may be configured to receive and store customer method customizations, which includes one or more modifications that can be saved as a customized or modified method. The customer can access the customized method use in a repeatable fashion within the market research platform.

In an embodiment, the market research system can implement customized and non-customized data collection methods. Non-customized methods may be referred to as standard methods. Customized methods may be generated by modifying non-customized methods. In some cases, non-customized methods may be used only “as is” with no changes to the underlying composition. In an embodiment, the market research system includes a method builder module configured to generate a customized method based on received customization data. The method builder module may replace customization tasks otherwise performed by humans, which may reduce time in building the customized method from hours or days to minutes.

The market research system may provide deeper automation in both audience selection and reporting. The application of machine learning techniques can produce faster insights. Examples of insights can include ‘levels of interest” and “an emotional response” prediction.

The market research system may automate the process of gathering consumer opinions. Automation of the consumer opinion gathering may reduce time requirements (e.g. from weeks to hours).

Timing may be reduced, for example, by performing planning and setup of the data collection method automatically. This may include, for example, automatically performing such tasks as setting an audience (for the data collection method), writing the questions (for the data collection method), and programming the survey. In an embodiment, the end user (e.g. a customer) may select a few options (e.g. for customizing the data collection method, via a user interface of the system), adds their own stimuli, and launch the data collection method immediately. By doing so, the market research system may automate the work required to create and launch a survey (or other data collection method) that is currently performed by a human.

Existing approaches to market research can be time consuming and inefficient because a human agent is needed to perform various tasks such as planning the research, writing the survey, contacting a sample provider to access a sample, programming the survey, launching and managing the sample, packaging up the data at the end, and creating a visual report. Advantageously, various embodiments of the market research system can automate and perform some or all of the foregoing tasks such as planning the research, writing the survey, generating a sample, programming the survey, launching and managing the sample, packaging up the data at the end of the survey, and creating a visual report of the survey results.

In an embodiment, the system automatically defines a sample (respondents), writes and programs the survey, and generates a report based on results data (corresponding to responses received from the sample). The report may be in the form of a template that is stored and modified (and displayed) based on the results data for the survey. Generating the report may include generating and displaying insights derived from the results data. The system may implement an algorithm for scoring the results data, generating insights based on the output of the algorithm, and displaying the insights in report format.

The system may leverage tools that use machine learning platforms to simulate the responses that a human might provide when presented with a question or stimuli. For example, the system may create and or make accessible virtual customers based on a brand's customer segmentation studies. The system can then use machine learning to gradually build out a range of interests or preferences. The system can then test new messaging or ideas (e.g. in text form) against the interests of dozens of virtual customers in real-time.

The market research system may deliver consumer insights instantly via machine learning. The system may include or otherwise make accessible (e.g. through connecting to) a machine learning platform for teaching and expanding the interests of virtual customers by having the virtual customers “react” to content (articles, posts, transcriptions of audio or video content) sourced from the internet (e.g. internet content sourced 24 hours a day, 7 days a week).

The market research system may include a flexible and customizable data collection method that allows for customization of questions and reporting while automating labour intensive parts of the process.

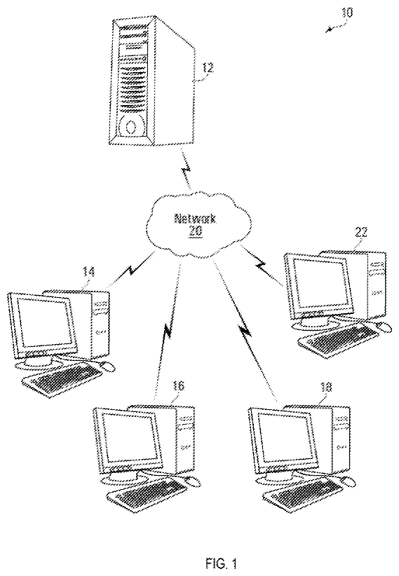

Referring now to FIG. 1, shown therein is a block diagram illustrating a system 10, according to an embodiment.

The system 10 may be an automated market research system. The automated market research system may provide market intelligence and consumer insights to a user, such as a market researcher. The market researcher may be a business decision maker at an organization, such as an executive, marketing professional, product manager, consumer insight professional, or the like.

The system 10 includes a research automation server platform 12, which communicates with a plurality of market researcher devices 16 and a plurality of research participant devices 18 via a network 20. The server platform 12 may also communicate with one or more additional servers such as technology layer server 14 and service layer server 22, which may include software components accessible to server 12 to enhance the functionality of the research automation server 12. The software components provided by servers 14 and 22 may enhance automation and/or virtualization of the system 10, which may increase speed and effectiveness of the market research process.

The server platform 12 may be a purpose-built machine designed specifically for providing a research automation platform. The server platform 12 may include multiple servers and tools to allow for automation.

The server 12 receives input data from the researcher device 16. The input data may include data collection method customization selections for customizing a data collection method based on the preferences or needs of the researcher.

The server 12 generates a market research project (e.g. survey, concept test, or the like) implementing a data collection method based on the input data received from the researcher device 16. The market research project may be a customized data collection method.

The server 12 may send data to and receive data from the participant device 18 or servers 14, 22 in the execution of the project and collection of response data.

Response data includes a participant's response to market research data, such as marketing assets (e.g. images, video), presented via the data collection method. The response data may include response data received from the participant device 18 and/or analyzed response data from the participant device 18 or the servers 14, 22.

In some cases, the research participant at the participant device 18 may not be a human but may be an automated or virtual persona. The virtual persona may represent a plurality of individual participants or respondents, which may improve efficiency of data collection. Where the participant is automated or virtual, the participant device 18 may be the server 14 or 22.

The server 12 may deliver project data (i.e. the customized data collection method or market research project) to the participant device 18 and receive response data therefrom automatically and without human input. The response data may be raw response data from participants (e.g. answers to questions) or may be analyzed response data generated at the participant device 18 via analysis performed on the response data by one or more software components located at or otherwise accessible to the participant device 18.

The server 12 may store data collection method template data representing a plurality of data collection methods (e.g. survey, concept test, user experience test, value proposition test) that can be customized by the researcher by making selections and/or providing input data at the researcher device 16.

Method customization data corresponding to selections and input provided by the researcher via the researcher device 16 can be stored at server 12. Method customization data is used to create a custom data collection method project for the researcher and may include any one or more of method selection data (e.g. a data collection method selection), audience selection data, and market research data (e.g. marketing assets to be tested, survey questions).

Response data can also be stored at server 12. Response data may include participant response data, analyzed response data, and report data generated therefrom.

Generally, in an embodiment, the researcher interacts with the system 10 via a user interface provided at the researcher device 16. The researcher can select and/or input various data collection method customizations. Based on the received method customization data, the server 12 can generate a customized data collection method or “project” that can be used to collect market intelligence and consumer insights that are of value to the researcher.

Generating the customized data collection method may include accessing and/or linking to software components located at the server 12 or servers 14, 22 (e.g. which may be controlled by an API) that are configured to increase automation and/or virtualization of the data collection method.

The customized data collection method can be stored at the server 12 and accessed by a participant via a user interface provided at the participant device 18.

In cases where the participant is not a human (i.e. a virtual participant such as an automated or virtual persona), the interface may be an API or other such interface configured to coordinate communication and data transfer between the software component implementing the virtual participant and the software component implementing the data collection method (i.e. provide response data).

The participant can review the custom data collection method and provide response data via the user interface (or other interface, as the case may be). The response data can be sent from the participant device 18 to the server 12 without analysis or may be analyzed prior to sending. Analysis may include the application of machine learning or artificial intelligence techniques to the response data.

The response data received at the server 12 can be provided to an analysis engine which may include software components (e.g. ML or AI models or engines) that are located at the server 12 or that are otherwise accessible to the server 12 such as via an API (e.g. at server 14 or 22). The analysis engine generates analyzed response data, which may include or be further processed to provide consumer insights to the researcher.

The analyzed response data may be provided to a reporting engine. The reporting engine generates report data from the analyzed response data. The report data may include one or more data visualizations or presentations that may highlight key insights that are relevant to the researcher and present them in a way that is easily understood. The reporting engine may use various data visualization techniques to present the response data, such as text, images, dynamic visualizations, graphs, charts, tables, or the like. Report data can be sent from the server 12 to the user interface at the researcher device 18, where the report data can be rendered into a display such as by a report rendering module or the like.

The server platform 12, devices 16, 18 and servers 14, 22 may be a server computer, desktop computer, notebook computer, tablet, PDA, smartphone, or another computing device. The devices 12, 14, 16, 18, 22 may include a connection with the network 20 such as a wired or wireless connection to the Internet. In some cases, the network 20 may include other types of computer or telecommunication networks. The devices 12, 14, 16, 18, 22 may include one or more of a memory, a secondary storage device, a processor, an input device, a display device, and an output device. Memory may include random access memory (RAM) or similar types of memory. Also, memory may store one or more applications for execution by processor. Applications may correspond with software modules comprising computer executable instructions to perform processing for the functions described below. Secondary storage device may include a hard disk drive, floppy disk drive, CD drive, DVD drive, Blu-ray drive, or other types of non-volatile data storage. Processor may execute applications, computer readable instructions or programs. The applications, computer readable instructions or programs may be stored in memory or in secondary storage, or may be received from the Internet or other network 20.

Input device may include any device for entering information into device 12, 14, 16, 18, 22. For example, input device may be a keyboard, key pad, cursor-control device, touch-screen, camera, sensors (e.g. atmospheric, biometric, neuro, etc.) or computer microphone embedded in any type of digital device (such as a smartphone, computer, or a voice assistant). Display device may include any type of device for presenting visual information. For example, display device may be a computer monitor, a flat-screen display, a projector or a display panel or a voice-enabled device. Output device may include any type of device for presenting a hard copy of information, such as a printer for example. Output device may also include other types of output devices such as speakers, for example. In some cases, device 12, 14, 16, 18, 22 may include multiple of any one or more of processors, applications, software modules, second storage devices, network connections, input devices, output devices, and display devices.

Although devices 12, 14, 16, 18, 22 are described with various components, one skilled in the art will appreciate that the devices 12, 14, 16, 18, 22 may in some cases contain fewer, additional or different components. In addition, although aspects of an implementation of the devices 12, 14, 16, 18, 22 may be described as being stored in memory, one skilled in the art will appreciate that these aspects can also be stored on or read from other types of computer program products or computer-readable media, such as secondary storage devices, including hard disks, floppy disks, CDs, or DVDs; a carrier wave from the Internet or other network; or other forms of RAM or ROM. The computer-readable media may include instructions for controlling the devices 12, 16, 18, 22 and/or processor to perform a particular method.

Devices such as server platform 12 and devices 14, 16, 18 and 22 can be described performing certain acts. It will be appreciated that any one or more of these devices may perform an act automatically or in response to an interaction by a user of that device. That is, the user of the device may manipulate one or more input devices (e.g. a touchscreen, a mouse, a button, a sensor (e.g. atmospheric, biometric, neuro)) causing the device to perform the described act. In many cases, this aspect may not be described below, but it will be understood.

As an example, it is described below that the devices 12, 14, 16, 18, 22 may send information to the server platform 12. For example, a user using the device 18 may manipulate one or more inputs (e.g. a mouse and a keyboard) to interact with a user interface displayed on a display of the device 18. Generally, the device may receive a user interface from the network 20 (e.g. in the form of a webpage). Alternatively or in addition, a user interface may be stored locally at a device (e.g. a cache of a webpage or a mobile application).

Server platform 12 may be configured to receive a plurality of information, from each of the plurality of devices 14, 16, 18, 22.

In response to receiving information, the server platform 12 may store the information in a storage database. The storage may correspond with secondary storage of the devices 14, 16, 18 and 22. Generally, the storage database may be any suitable storage device such as a hard disk drive, a solid state drive, a memory card, or a disk (e.g. CD, DVD, or Blu-ray etc.). Also, the storage database may be locally connected with server platform 12. In some cases, storage database may be located remotely from server platform 12 and accessible to server platform 12 across a network for example. In some cases, storage database may comprise one or more storage devices located at a networked cloud storage provider.

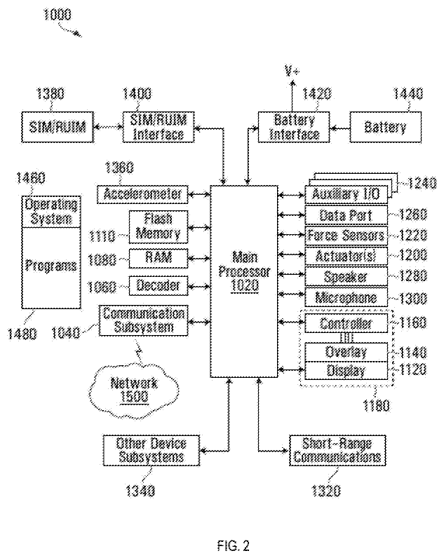

FIG. 2 shows a simplified block diagram of components of a device 1000, such as a mobile device or portable electronic device. The device 1000 includes multiple components such as a processor 1020 that controls the operations of the device 1000. Communication functions, including data communications, voice communications, or both may be performed through a communication subsystem 1040. Data received by the device 1000 may be decompressed and decrypted by a decoder 1060. The communication subsystem 1040 may receive messages from and send messages to a wireless network 1500.

The wireless network 1500 may be any type of wireless network, including, but not limited to, data-centric wireless networks, voice-centric wireless networks, and dual-mode networks that support both voice and data communications.

The device 1000 may be a battery-powered device and as shown includes a battery interface 1420 for receiving one or more rechargeable batteries 1440.

The processor 1020 also interacts with additional subsystems such as a Random Access Memory (RAM) 1080, a flash memory 1100, a display 1120 (e.g. with a touch-sensitive overlay 1140 connected to an electronic controller 1160 that together comprise a touch-sensitive display 1180), an actuator assembly 1200, one or more optional force sensors 1220, an auxiliary input/output (I/O) subsystem 1240, a data port 1260, a speaker 1280, a microphone 1300, short-range communications systems 1320 and other device subsystems 1340.

In some embodiments, user-interaction with the graphical user interface may be performed through the touch-sensitive overlay 1140. The processor 1020 may interact with the touch-sensitive overlay 1140 via the electronic controller 1160. Information, such as text, characters, symbols, images, icons, and other items that may be displayed or rendered on a portable electronic device generated by the processor 102 may be displayed on the touch-sensitive display 118.

The processor 1020 may also interact with an accelerometer 1360 as shown in FIG. 1. The accelerometer 1360 may be utilized for detecting direction of gravitational forces or gravity-induced reaction forces.

To identify a subscriber for network access according to the present embodiment, the device 1000 may use a Subscriber Identity Module or a Removable User Identity Module (SIM/RUIM) card 1380 inserted into a SIM/RUIM interface 1400 for communication with a network (such as the wireless network 1500). Alternatively, user identification information may be programmed into the flash memory 1100 or performed using other techniques.

The device 1000 also includes an operating system 1460 and software components 1480 that are executed by the processor 1020 and which may be stored in a persistent data storage device such as the flash memory 1100. Additional applications may be loaded onto the device 1000 through the wireless network 1500, the auxiliary I/O subsystem 1240, the data port 1260, the short-range communications subsystem 1320, or any other suitable device subsystem 1340.

For example, in use, a received signal such as a text message, an e-mail message, web page download, or other data may be processed by the communication subsystem 1040 and input to the processor 1020. The processor 1020 then processes the received signal for output to the display 1120 or alternatively to the auxiliary I/O subsystem 1240. A subscriber may also compose data items, such as e-mail messages, for example, which may be transmitted over the wireless network 1500 through the communication subsystem 1040.

For voice communications, the overall operation of the portable electronic device 1000 may be similar. The speaker 1280 may output audible information converted from electrical signals, and the microphone 1300 may convert audible information into electrical signals for processing.

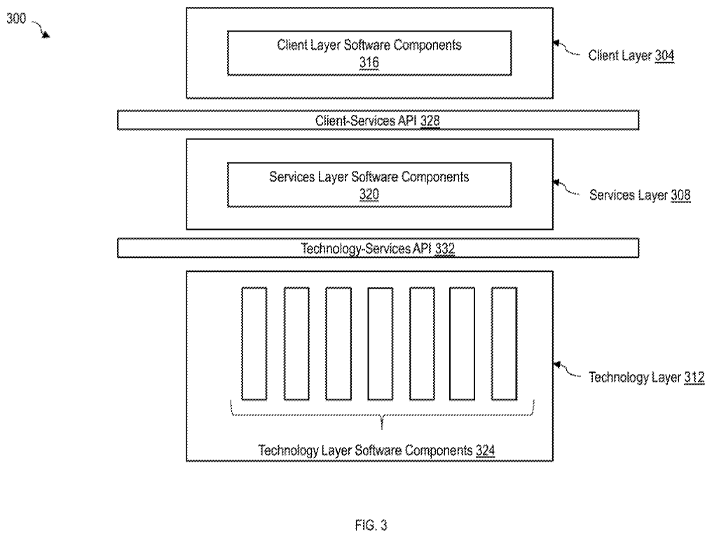

Referring now to FIG. 3, shown therein is a layered architecture 300 implemented by the automated market research system 10 of FIG. 1, according to an embodiment.

Each of the layers in the layered architecture 300 includes one or more software components that work together to provide functionality of the system 10.

Software components in a given layer may be configured to communicate (transfer and receive data) with software components in the layer above or below in the layered architecture 300.

Software components may include a plurality of software modules including computer-executable instructions that, when executed by a processor, cause one or more computing devices (such as devices 12, 14, 16, 18, 22 of FIG. 1) to perform certain actions and provide certain functionalities described herein.

The layered architecture 300 includes a client layer 304, a services layer 308, and a technology layer 312. Each of the layers 304, 308, 312 may include a plurality of software components.

The software components include client layer software components 316, services layer software components 320, and technology layer software components 324.

The software components 316, 320, 324 may be configured to perform a market research automation or virtualization function. By automating or virtualizing processes, the system 10 may facilitate quicker market research data collection and response data analysis.

Technology layer software components 324 may be configured to interact and communicate with software components in the services layer 308.

Services layer software components 320 may be configured to interact and communicate with technology layer software components 324 and client layer software components 316.

Client layer software components 316 may be configured to interact and communicate with services layer software components 320.

In a particular case, client layer software components 316 may be configured to perform functions that may otherwise performed by services layer software components 320 or to automate or virtualize certain functions of the system 10 that may obviate software components that may otherwise be included in the services layer 308. In such a case, client layer software components 316 may be considered to communicate or interact directly with technology layer software components 324.

In a particular advantage of the present disclosure, the system 10 may, in an embodiment, be designed to reduce or compress the services layer 308. This may include adding or moving software components to the client layer 304. This may include automating or virtualizing various processes using software components in the client layer 304 and facilitating communication between software components via specifically designed interfaces (e.g. API). This may be particularly advantageous to improving the market research process as various software components in the services layer 308 are typically provided by different providers and may include different interfaces leading to increased complexity of access and use.

By compressing the services layer 308 through adding or moving software components to the client layer 304 that automate or virtualize aspects of the market research process and system 10, researchers can access and use custom data collection methods that incorporate a variety of market research technology tools through a consistent user interface. The researcher can interact with the system 10 using the user interface at the researcher device 18, which may simplify the market research process for the researcher while providing access to valuable market research tools and technologies provided by technology layer software components 324 that may generate better insights and provide faster results.

The layered architecture 300 includes a client-services application programming interface (“API”) layer 328. The client-services API layer 328 includes one or more APIs configured to facilitate interaction and communication between client layer software components 316 and services layer software components 320.

The APIs in the API layer 328 may manage or specify how client layer software components 316 and services layer software components 320 interact. Typically, a client layer software component 316 may interact with a services layer software component 320 by making a request of the services layer software component 320 and receiving a response therefrom. Similarly, a services layer software component 320 may make a request to and receive a response from a client layer software component 316. The API may specify the format of the request and response.

The layered architecture 300 includes a services-technology API layer 332. The services-technology API layer 332 includes one or more APIs configured to facilitate interaction and communication between services layer software components 320 and technology layer software components 320. The API layer 332 functions similarly to the API layer 328 but for interactions between the services and technology layer software components 320, 324.

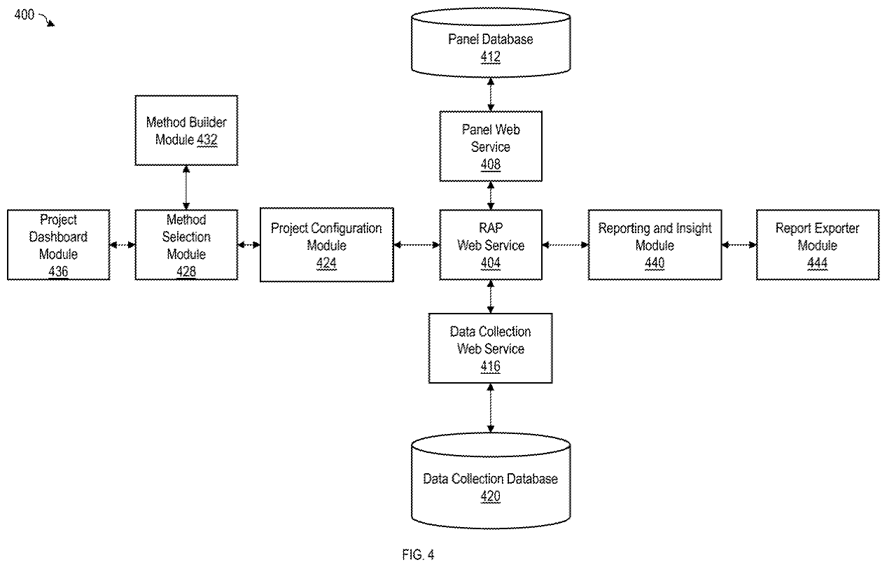

Referring now to FIG. 4, shown therein is a system framework 400 for a market research system, according to an embodiment. The framework may be a framework for the market research system 10 of FIG. 1.

The framework 400 includes a plurality of software components. The framework 400, or a portion thereof, may be implemented by research automation server 12 of FIG. 1.

Components of the framework 400 may be implemented at any one or more of servers 14, 22 or devices 16, 18 of FIG. 1, in communication with server 12.

The framework 400 and components thereof may be implemented in one or more layers of the layered architecture 300 described in FIG. 3.

The framework 400 includes a research automation platform (RAP) web service 404. The RAP web service 404 controls actions between the client and the system. The RAP web service 404 includes an API that is called by internal and client interactions to create, configure, launch and report on projects.

The framework 400 includes a panel web service 408. The panel web service 408 is an API that allows the RAP to connect with a panel database (e.g. panel database 412, below) to request a sample to complete surveys. The panel web service 408 is used to automate the survey fielding process. This may advantageously reduce survey fielding time and effort.

The framework 400 includes a panel database 412. The panel web service 408 connects to and provides access to the panel database 412. The panel database 412 may contain the information associated with panel members that is used by audience selection interface to define, assemble, and connect the right people to make up the sample. The panel database 412 stores survey respondent information. The RAP connects to the panel database 412 to retrieve the correct respondents for the survey so the project can collect the completes required.

The framework 400 includes a data collection web service 416. The data collection web service 416 is an API that is used to create surveys, configure surveys, and retrieve survey data.

The framework 400 includes a data collection database 420. The data collection web service 416 connects to and provides access to the data collection database 420. The data collection database 420 contains the data collected through data collection projects.

The framework 400 includes a project configuration module 424. The project configuration module 424 may include a plurality of software tools for customizing a data collection method.

Customizing the data collection method may include adding custom questions to a project or method template. The customizations may be selected or otherwise indicated by the researcher at the researcher device 16.

The project configuration module 424 may allow the customer to setup a market research project (including a data collection method). This may include naming the project, confirming an audience, selecting one or more geographical regions, or selecting one or more languages. This may also include uploading stimuli and inputting any variables required by the data collection method. The project configuration module 424 can be used to configure the project to fit the needs of the market researcher. The market researcher user may input and upload settings to customize the project.

The framework 400 includes a method selection module 428. The method selection module 428 may include or otherwise communicate with a virtual recommendation engine. The virtual recommendation engine may be located at the server 12. The virtual recommendation engine may be located at server 14 or server 22 and be accessible to the server 12, for example via an API.

The method selection module 428, through accessing and providing data to the virtual recommendation engine, may act as a type of “research concierge” that can assist a researcher in selecting an appropriate data collection method.

The method selection module 428 may receive researcher needs data. The researcher needs data is provided by the researcher user via the user interface at the researcher device 16. The researcher needs data may include answers to a plurality of questions that can be used by the virtual recommendation engine to generate method suggestion data.

The method suggestion data can be provided to the user interface at the researcher device 16 where it can be presented to the researcher. The method suggestion data may include one or more suggested data collection methods and/or more general information about appropriate methods and method selection.

The method selection module 428 may use a chat bot trained to ask questions, receive answers, and determine a method suggestion. The chat bot (or virtual engine) may be located at the server 12 or server 14 or 22 and accessible through an API or the like.

The framework 400 includes a method builder module 432. The method builder module 432 includes a method creation tool that allows for quick and easy configurations for flexible and customized data collection methods. Method builder module 432 may allow customers to do advanced customization to the audience selection, questions asked, weighing of answers that affect how the insights are communicated.

The method builder module 432 is configured to receive method customization data. The method customization data is provided by the researcher at the user interface of researcher device 18. The method customization data may include configurations to existing data collection method templates stored at server 12.

The method customization data and method template data can be used by the method builder module 432 to generate the custom data collection method. The customer data method can be stored as a project at the server 12, where it may be accessed via a researcher participant via an interface provided at the researcher device (e.g. project dashboard generated by dashboard module 436 described below).

The framework 400 includes a project dashboard module 436. The project dashboard module 436 renders and displays a project dashboard.

The project dashboard module 436 is configured to receive various project data associated with a researcher account and display the project data. Project data may include projects (e.g. customized data collection methods) already created by the researcher using the system 10 and various project metadata for the projects such as a creation date, status, responses, and the like.

The dashboard module 436 may provide an interface through which the user can create projects and access created projects. The dashboard module 436 may receive project data from the method builder module 432 corresponding to a created custom method.

The project dashboard module 436 may be configured to receive input data from the user requesting certain project data to be displayed. The project dashboard module 436 may display different project data based on received input data. The input data received via the project dashboard module 436 may be used by other modules in the framework in performing certain functionalities.

Components 436, 428, 432 and 424 allow the user to navigate to different pages by using parameters in the URL. Components 424 and 404 may exchange data frequently to support functionality such as creating projects, uploading assets, previewing a survey experience, and data collection database creation.

The framework 400 includes a reporting and insight module 440. The reporting and insight module 400 generates report data from response data stored at the server 12. The report data includes insights. Insights may include a winner selected from several concepts being tested by in the data collection method, highlighting where significant differences have been detected between tested concepts, an average of all concepts tested using the method, or outliers presented for consideration.

The reporting and insights module 440 may include reporting tools such as banners and weights. Banners enable answer data to be sliced by other variables such as demographics like age, gender, region, etc. The reporting and insights module 440 may allow researchers to derive better insights from projects. This may be accomplished, for example, by automating the display of data for better interpretation and the uncovering of insights (as discussed above). The reporting and insights module 440 may utilize machine learning techniques for analyzing response data and generating report data.

The reporting and insight module 440 may be configured to process data. Data processed by the reporting and insights module 440 may include survey response data or may include data collected through technologies such as chatbots, facial coding, eye-tracking, biometrics or neuro sensors. This may allow the identification of additional insights such as trends and outliers. Features such as banners allow a customer to manipulate the data to uncover insights. By clicking a few buttons to manipulate the data a researcher may be able to do in minutes what could take hours and days to explore.

The reporting and insight module 440 may integrate virtualization into reporting. In some cases, the audience may be virtualized. The audience may be virtualized by creating a simulated model of a customer segment. Messages and ideas can be tested against the simulated model of a customer segment in real-time.

The reporting and insight module 440 generates and presents instant insights and data. The insights and data may be generated through the use of automated personas, emotional analysis, or other automation and virtualization techniques. Emotional analysis may include using an AI system that is trained to simulate and estimate a human's emotional response to stimuli such as text or images. These tools may generate feedback and insights in real time as the stimuli is being tested against an AI-trained system rather than humans. This may provide an improvement, allowing researchers to test more ideas earlier in the process at a scale that is not possible when engaging with human respondents. Human respondents can be used to review an optimized group of messages or ideas. The simulated models may be continuously updated through exposure to new trends and updated news and interests.

The framework 400 includes a report exporter module 444. The report exporter module 444 is configured to receive report data in a first format from the reporting and insight module 400 and generate exported data by converting the report data into a second format. The second format may be a format more suitable for display and consumption by the user researcher. Report exporter module 444 may allow the researcher to download the report data in a number of forms. Forms may include raw data, or formatted as a PowerPoint, PDF, or the like.

The report exporter module 444 can convert the report data into a PowerPoint, PDF or other suitable format. PDF may be easier to view and distribute, while PowerPoints may align with a tool the researcher may be using to create their final report.

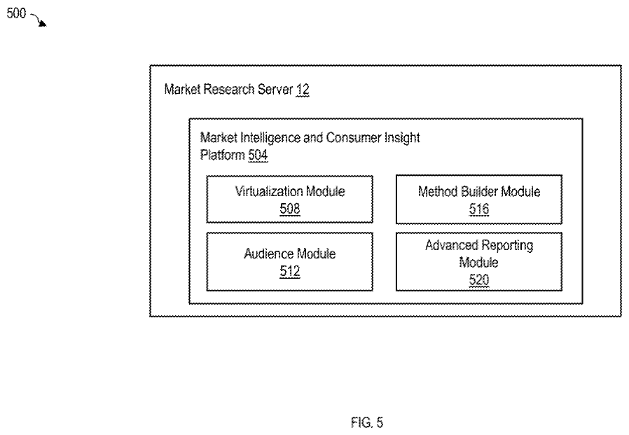

Referring now to FIG. 5, shown therein are software components 500 of market research system 10 of FIG. 1, according to an embodiment.

Software components 500 may be implemented at server 12 of FIG. 1. In some cases, software components 500, or portions thereof, may be implemented at researcher device 16 and/or servers 14, 22 of FIG. 1.

The software components 500 may provide market intelligence and consumer insights to the user.

The software components 500 may automate the gathering of real consumer opinions. In doing so, the software components 500 may reduce time to collect and analyze such consumer opinions, for example from weeks to hours. Advantageously, the software components 500 may be configured to integrate multiple market research technologies into a single platform.

The system 500 may automate complex research methods faster and easier. Automation may include, for example, any one or more of automated audience selection, automated data collection technique (e.g. no survey programming), and automated reporting.

The system 500 includes a market intelligence and consumer insights platform 504. The platform 504 may be faster and more flexible than existing research platforms. This may be achieved by automating certain customization tasks (for customizing a project for a researcher) that may otherwise be manually performed by a developer.

The platform 504 uses automation and machine learning to provide user access to proven research methods. In an embodiment, an AI-trained system provides simulated feedback in real-time. This may provide an improvement over similar feedback being provided through a group of human respondents. The platform 504 delivers consumer insights instantly via machine learning. Machine learning is used during the training of the AI components of the system that are used to deliver real-time feedback to research automation platform users.

The platform 504 may allow a non-developer user to customize research methodologies and technology integrations less time than current solutions (e.g. in minutes instead of days).

The software components 500 include a virtualization module 508.

The virtualization module 508 implements a virtual researcher (or research concierge) for making data collection method recommendations, identifying insights, and validating concepts. The virtualization module 508 is trained to look for and report on patterns in the data, compare against past averages and outliers. The virtual researcher may perform these functions using automated personas and emotional analysis. In cases of the virtual personas, the concierge runs the stimuli against simulated customer segments and receives feedback instantly.

The virtualization module 508 may be configured to receive data defining researcher needs and generate a method recommendation based on the researcher needs data. In an example, a customer inputs their business challenge—based on historical data of which research method should return the data and insights best suited for helping the customer make that business decision—the virtual research makes a recommendation of which method to use . . . and then can execute the project for the user with their approval of the recommendation.

The virtualization module 508 is configured to receive project data. The project data includes Information associated with the audience selection, the survey questions and responses, the logic of the survey. The virtualization module 508 may also be configured to generate insights from the project data. The Insights may include best concept, the ranking of ideas, how well an idea resonates, etc. The insights may be generated by an algorithm of virtualization module 508.

The virtualization module 508 may implement a simulated emotion response presentation.

The virtualization module 508 may implement a digital twins function.

The virtualization module 508 may reduce time for providing results data. The virtualization module 508 virtualizes or automates by executing on a set of rules on how best to carry out a research study based on a customer's business challenge. The virtualization module 508 automates the things that would normally have to be done by a human project manager. For example, the virtualization module 508 may facilitate the provision of results data in minutes instead of hours or days.

The software components 500 include an audience module 512. The audience modules 512 allows a researcher to specify an audience for a project. The audience includes the participants providing the participant response data.

The audience module 512 may be configured to receive an audience specification or selection from a researcher via a user interface at the researcher device 16.

The audience module 512 may generate pricing data, timeline data and feasibility data from the audience specification. The audience module 512 may provide such data to the user in real time.

The audience specification can be used to automate the project settings. The Project settings may include an audience selection or the programming of survey logic. By automating the project settings, the audience module 512 may remove programmer and project manager participation (by automating tasks otherwise performed by them) and increase speed and efficiency of the process.

The audience module 512 includes a panel API integration.

The audience module 512 may reduce client/sales touch points (by automating tasks otherwise performed by humans). The audience module 512 may provide automation for faster launch and fielding. Launch and fielding include connecting an audience through one or more panel partners and managing the activities of a project when the project is live and when the project closes and the data is made available to the researcher.

The software components 500 include a method builder module 516. The method builder module 516 is a software tool that can create repeatable methods that include one or more customizations to a data collection method template. The method builder module 516 may allow a user (e.g. a developer) to make changes to aspects of the data collection method such as the audience, survey questions, weighting of the questions, changes to reporting, or the like. Once made, these changes can be saved as a repeatable method that the researcher can use at any time.

The method builder module 516 may reduce onboarding time for new users, for example from weeks to hours. Onboarding time refers to the time to get a new customer set up on the research automation platform with customized versions of methods best suited to their organization's research goals. While developers may perform similar customization now, the process takes time and doesn't scale well. Using automation as described can speed the process up, which may get a research using the customized method quicker, allowing insights to be received by the researcher sooner.

The method builder module 516 may provide faster integrations with partners and technologies, for example from weeks to days. By creating standard APIs and SDKs that technical partners can use to build and connect their technology into the research automation platform, we can reduce time and resources required to do this work.

The method builder module 516 can generate a flexible and customizable data collection method for users. The method builder module 516 may allow a non-developer user to create a custom data collection method that can be used to gather response data. The method builder module 516 may allow for custom questions and reporting while automating the entire process. Researchers may need the ability to create their own Methods to complete a project. Researchers may need to reuse their own customer methods. Researchers may want to share their custom Methods within their organization. Researchers may want to use an existing Method but provide some customization for their specific needs (editing questions and answer options, or adding new questions) without the need for custom development.

The method builder module 516 may be configured to customize data collection method properties based on method customization data provided by a user. The data collection method properties may include any one or more of functionality (e.g. rules by which the method's data collection activities are conducted), pricing, timing and integrations (e.g. any partner technologies that may be used as part of the method). The audience may be customizable based on size or demographics such as age, region, gender, customer vs non-customer, etc. The method builder module 516 may customize the data collection method faster than existing solutions, for example in hours instead of days.

The software components 500 include an advanced reporting module 520. The advanced reporting module 520 receives response data and analyzes the response data. Response data may be analyzed based on the rules built into the algorithm which scores the data to be displayed as charts or other types of visualizations. The reporting module 520 is configured to receive, accept, and process any type of data. The reporting module 520 can accept quantitative and qualitative data which is structured and unstructured. The reporting module 520 also accepts data from 3rd party technologies which are parsed and structured into a readable format. The advanced reporting module 520 may use any one or more of weights, banners, crosstabs, and trackers. These things allow for the data to be manipulated and compared to uncover additional insights. The advanced reporting module 520 may generate and display additional insights that can be saved and exported (e.g. via exporter module 444 of FIG. 4).

The system may compress the services layer 318 (or portion thereof) so that it is automated. Virtualization may be used.

In an example, Voxpopme or a similar application may be used by the system to provide automation. Voxpopme includes functionality to collect video responses and process the audio into a text transcription and analyze the content of the transcriptions. Voxpopme can also automate the creation of compilation video from responses based on identified themes, sentiments, or the like.

In an example, CRIS or a similar application may be used by the system to provide virtualization. CRIS may take the methods that a human interviewer may use to conduct an interview with a person and automates the approach. By automating the approach, interviews may be conducted at a significantly increased scale.

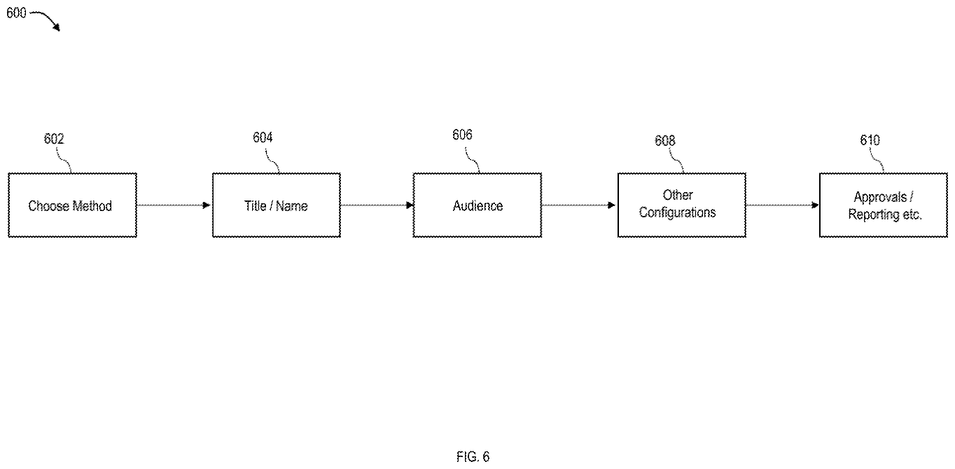

Referring now to FIG. 6, shown therein is a research automation method flow 600, according to an embodiment. The method flow 600 may be implemented by the automated market research system 10 of FIG. 1. For example, aspects of the method flow 600 may be implemented by the research automation server platform 12 and the market researcher device 16 of FIG. 1, or as part of the layered architecture 300 of FIG. 3. Certain modules described in FIG. 6 may include server-side software components (operating on a server device, such as server 12 of FIG. 1) and client-side software components (executing on a client device, such as devices 16, 18 of FIG. 1).

The method flow 600 includes a choose method module 602, a project title/name module 604, an audience module 606, an other configuration module 608, and an approvals/reporting module 610.

At 602, a user interface allowing a user to choose a data collection method is generated and displayed on a user device (e.g., market researcher device). The user interface may present multiple data collection method options. The method options may include standard or template data collection methods or a customized data collection method. The user interface is configured to receive input data indicating a method selection made by the user. The input data is sent to the server and may be used to retrieve the selected method for storage (e.g., data collection method database).

Receipt of input data indicating the method selection may initiate or invoke the project title/name module 604.

At 604, a user interface allowing a user to input a project title or name is generated and displayed on the user device. The user interface may include a text input interface, such as a text box, configured to receive input data indicating the project name. Once the project name input data is received by the user interface, the project name may be provided to the server and stored such that it is linked to the selected data collection method from 602.

Receipt of input data indicating the project title or name may initiate or invoke the audience module 606.

At 606, a user interface allowing a user to configure an audience is generated and displayed on the user device. The user interface may include a plurality of user interface elements configured to receive input data describing a particular characteristic of the audience. For example, the user interface may include user interface elements for the selection of one or more languages, one or more countries, a number of concepts (being tested using the audience), a number of responses, and one or more demographics. In each case, the user may provide input data to the user interface element indicating a selection that defines the audience. Accordingly, the received input data configuring the audience (audience configuration data) may include language selection data, country selection data, number of concepts data, number of responses data, and demographic data. Once the audience configuration data is received by the user interface, the audience configuration data may be provided to the server and stored such that it is linked to the method selection and the project name.

Receipt of the audience configuration data may initiate or invoke the other configurations module 608.

At 608, a user interface allowing a user to provide other method or project configurations is generated and displayed on the user device. The user interface may include a plurality of user interface elements configured to receive input data describing a particular configuration of the data collection method or project. For example, the user interface may include one or more user interface elements for receiving a text description of the data collection method (description data). The user interface may include one or more user interface elements for uploading a concept to be tested via the data collection method. The concept may be, for example, a media file such as a video clip or an image. Generally, the concept includes marketing content that the market researcher wants to test using the audience configured by the audience configuration data. The user interface may include one or more user interface elements configured to receive input data describing a custom attribute of the data collection method. The user interface may include one or more user interface elements configured to receive input data (e.g. a selection) indicating to include a technology layer software component (e.g., technology layer software component 324 of FIG. 3) in the data collection method. The technology layer software component may be accessed using an API (e.g., technology-services API 332 of FIG. 3). The technology layer software component may be a third-party software application. The technology layer software component may provide automation. The technology layer software component may include functionality to collect video responses and analyze the content of the video responses (e.g., Voxpopme). The user interface may include one or more user interface elements (such as a text box) for receiving input data of a PO number or a note.

Input data provided to the user interface may be provided to the server and stored such that the input data is linked to the data collection method, project, and audience configuration.

Receipt of the input data via the other configuration modules may initiate or invoke the approvals/reporting module 610.

At 610, a user interface allowing a user to approve a data collection method is generated and displayed on the user device. The user interface (or another user interface) may also allow a user to view reporting data. The reporting data includes data describing respondent interaction with the data collection method (e.g., response content, number of responses, etc.)

The user interface at 610 is configured to display the data collection method to the user for approval. The user interface may use various data provided by modules 602-608 to present the data collection method for approval. The user interface includes a user interface element for receiving input data indicating the data collection is approved/not approved. Receipt of input data approving the data collection method may cause the data collection method to go live (i.e., be made available to respondents, such as on device 18 of FIG. 1).

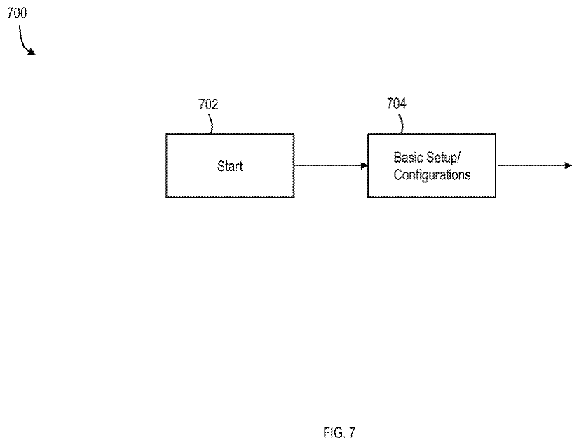

In some embodiments, the research automation platform of the present disclosure may be implemented according to a modular approach. The modular approach may provide users with more control and flexibility in creating and deploying data collection methods and may improve speed of gathering research insights for users. Such embodiments will now be described with reference to FIGS. 7 to 12, which illustrate method flows for a plurality of modules which may be used as part of the research automation platform. The method flows and modules described in FIGS. 7 to 12 may be implemented by the automated market research system 10 of FIG. 1. For example, aspects of the method flows may be implemented by the research automation server platform 12 and the market researcher device 16 of FIG. 1, or as part of the layered architecture 300 of FIG. 3. Certain modules described in FIGS. 7 to 12 may include server-side software components (operating on a server device, such as server 12 of FIG. 1) and client-side software components (executing on a client device, such as devices 16, 18 of FIG. 1).

Referring now to FIG. 7, shown therein is a method flow 700 for a basic configuration module, according to an embodiment. The method flow 700 may provide the starting or entry point for a user configuring a data collection method using the system.

At 702, the method flow 700 starts. The method flow 700 proceeds to a basic setup/configuration module 704.

At 704, a user interface allowing a user to input basic setup and configuration data is generated and displayed on a user device (e.g., market researcher device). The user interface may include one or more user interface elements for receiving input data of a user (e.g., selection, text input). The user interface may include a user interface element for receiving name data. The user interface may include a user interface element for receiving description data. The user interface may include a user interface element for receiving language data indicating a language selection. The user interface may include a user interface element for receiving brand/community data. The brand/community data may include a logo (e.g., of the market researcher) and colours. The user interface element may facilitate the upload of a logo.

The basic setup and configuration data received at 704 may be provided to the server and stored.

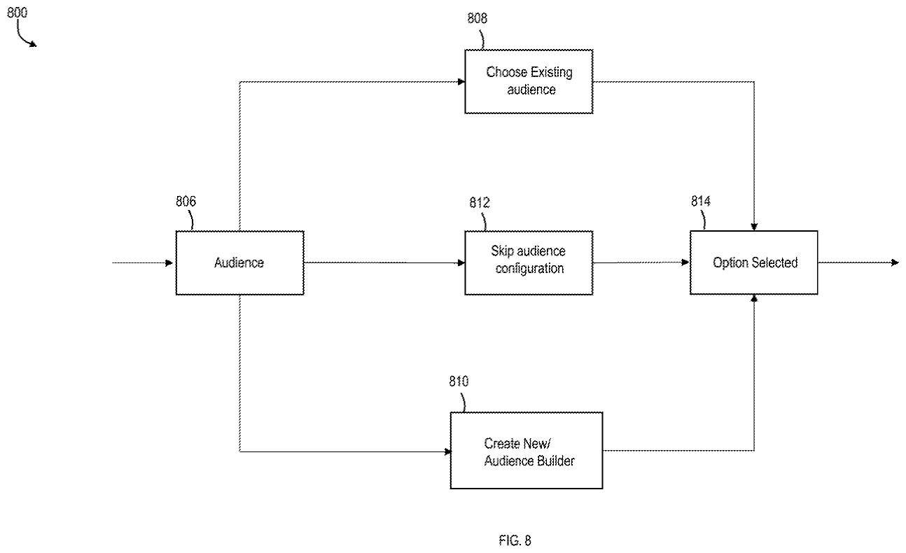

Referring now to FIG. 8, shown therein is a method flow 800 for an audience configuration module, according to an embodiment.

At 806, an audience configuration module is invoked. The audience configuration module is configured to generate a user interface displaying user interface elements allowing a user to select an existing audience 808, create a new audience 810, or skip audience configuration 812. The user interface elements may, for example, be selectable icons including text describing the option.

The choose existing audience 808 option may correspond to one or more existing audiences configured by the user and stored in association with a user account. Upon the user interface receiving input data indicating a choose existing audience 808 selection, the audience configuration module may be configured to retrieve one or more existing audiences of the user from an audience database or other storage and display the one or more existing audiences to the user via the user interface. For example, each existing audience may include a descriptor describing the existing audience that can be displayed. The descriptor allows the existing audience to be distinguished from other existing audiences. The descriptor may include additional data about the existing audience to provide further information about the existing audience to the user. The user interface may be configured to receive an existing audience selection from the user and proceed at 814.

Receipt of input data indicating a selection of the create new audience 810 may invoke an audience builder module. The audience builder module may include a user interface configured to present various audience configuration options to a user and receive input data indicating selections of various audience configurations (audience configuration data). The received audience configuration data may then be stored by the server as an existing audience. Upon receiving the audience configuration data for the new audience, the flow proceeds to 814.

The audience builder module may enable users to select multiple audience criteria (versus selecting a single demographic) in building an audience. By storing the audiences created using the audience builder module, users (market researchers) can reuse the built audience for multiple market research projects. This may advantageously allow market researcher users to target the same type of people/respondents (i.e., the reused audience) to test a new concept. The audience builder module may be used to generate an audience independent of creating a method. As a result, in embodiments where the system includes an audience builder module, the user may initiate a project from an audience (e.g., by building an audience using the audience builder module) or from a method.

Receipt of input data indicating a selection of the skip audience configuration 812 option causes the flow to proceed to 814.

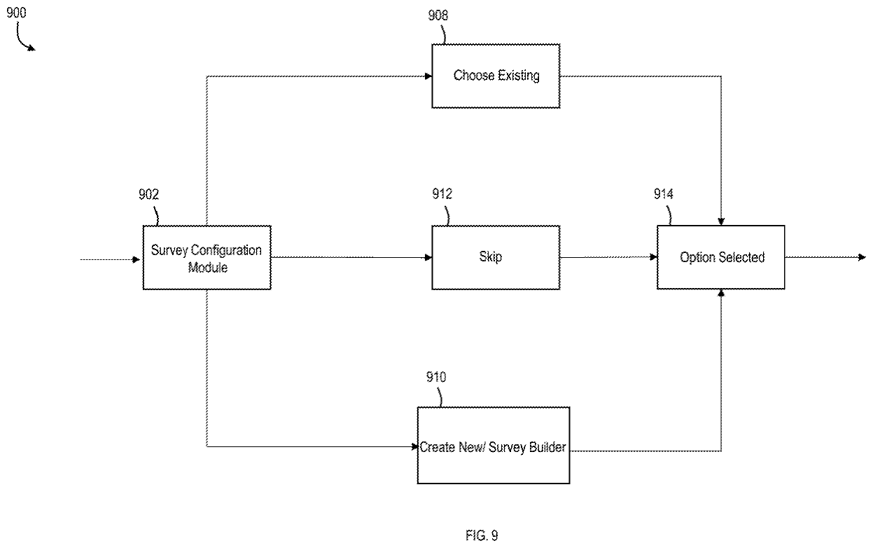

Referring now to FIG. 9, shown therein is a method flow 900 for a survey configuration module, according to an embodiment.

At 902, a survey configuration module is invoked. The survey configuration module is configured to generate a user interface displaying user interface elements allowing a user to select to an existing survey 908, create a new survey 910, or skip survey configuration 912. The user interface elements may, for example, be selectable icons including text describing the option.

The choose existing survey 908 option may correspond to one or more existing surveys configured by the user and stored in association with a user account. Existing surveys may have been created previously using the create new survey 910 option, such as described below. Upon the user interface receiving input data indicating a choose existing survey 908 selection, the survey configuration module may be configured to retrieve one or more existing surveys of the user from a survey database or other storage and display the one or more existing surveys to the user via the user interface. For example, each existing survey may include a descriptor describing the existing survey that can be displayed. The descriptor allows the existing survey to be distinguished from other existing surveys. The descriptor may include additional data about the existing survey to provide further information about the existing survey to the user. The user interface may be configured to receive an existing survey selection from the user and proceed at 914.

Receipt of input data indicating a selection of the create new survey 910 may invoke a survey builder module. The survey builder module may include a user interface configured to present various survey configuration options to a user and receive input data indicating selections of various survey configurations (survey configuration data). Survey configuration may include selecting an existing data collection method template and configuring the data collection method template according to input data provided by the user. The received survey configuration data may then be stored by the server as an existing survey. Upon receiving the survey configuration data for the new survey, the flow proceeds to 914.

Receipt of input data indicating a selection of the skip survey configuration 912 option causes the flow to proceed to 914.

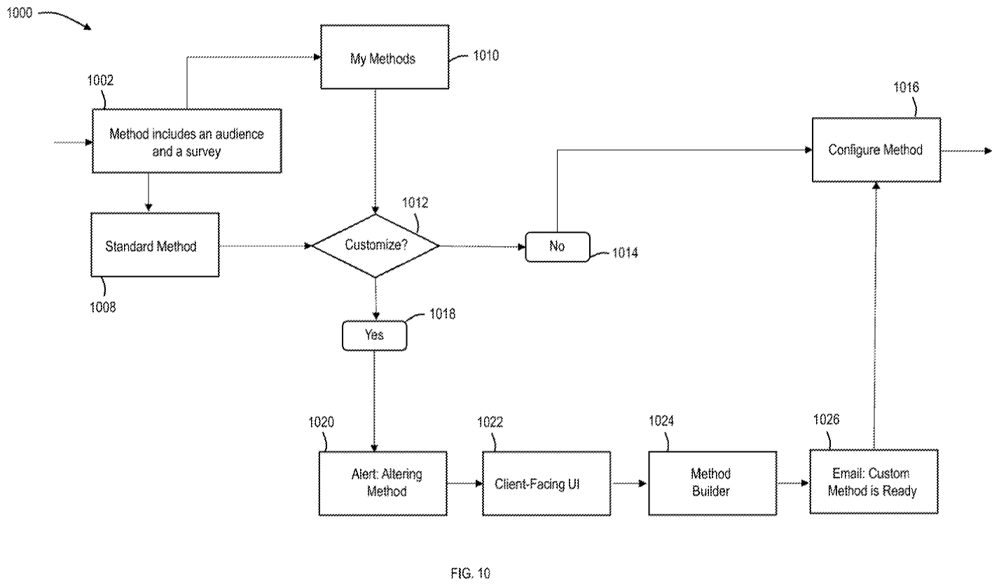

Referring now to FIG. 10, shown therein is a method flow 1000 for a method configuration module, according to an embodiment.

At 1002, a method configuration module is invoked. The method configuration module configures a data collection method. A method includes an audience and a survey. The audience component and survey component of the method may be configured by the audience configuration and survey configuration modules of FIGS. 8 and 9, respectively.

The survey configuration module is configured to generate a user interface displaying user interface elements allowing a user to select to a standard method 1008 or an existing method 1010 of the user (i.e. “my methods”). The user interface elements may, for example, be selectable icons including text describing the option.

A standard method is a pre-built method stored by the system. The pre-built method may be provided by the research automation platform provider. The standard method 1008 option may include a list of standard methods from which the user can input a selection.

An existing method 1010 corresponds to a method previously configured by the user and stored in association with the user's account. The existing method 1010 option may include a list of existing methods from which the user can input a selection.

Once the user has selected an existing method 1010 or a standard method 1008, the method configuration module presents an option via the user interface to customize the selected method type at 1012 (e.g. via a selection of yes or no).

At 1014, the user interface receives input data from the user indicating the user does not wish to customize the method.

Upon receiving the input data at 1014, the method configuration module invokes a configure method module 1016. The configure method module 1016 includes a user interface for receiving input data from the user configuring the selected method.

At 1018, the user interface receives input data from the user indicating the user wishes to customize the method and proceeds to 1020.

At 1020, the system generates an alert indicating the selected method is being altered. The user is notified to prevent accidental change to a method without being aware of how it affects the method and report.

At 1022, a client-facing UI is generated. The client-facing UI may be configured to display various customization options and receive input data indicating the selected customizations.

At 1024, the customization data provided at 1022 is provided to a method builder module. The method builder module may be an internal software tool. The method builder module customizes the method according to the customization data received at 1022. Options within the Method Builder module may include, for example, modifying or creating audience criteria, adding qualitative or quantitative technologies for deeper insight, and modifying or creating questions within a survey

At 1026, an electronic message is generated indicating the custom method is ready. The electronic message may be an email. The electronic message is provided to the user. After generation of the custom method, the flow proceeds to the configure method module at 1016, which may allow the user to further configure the customized method.

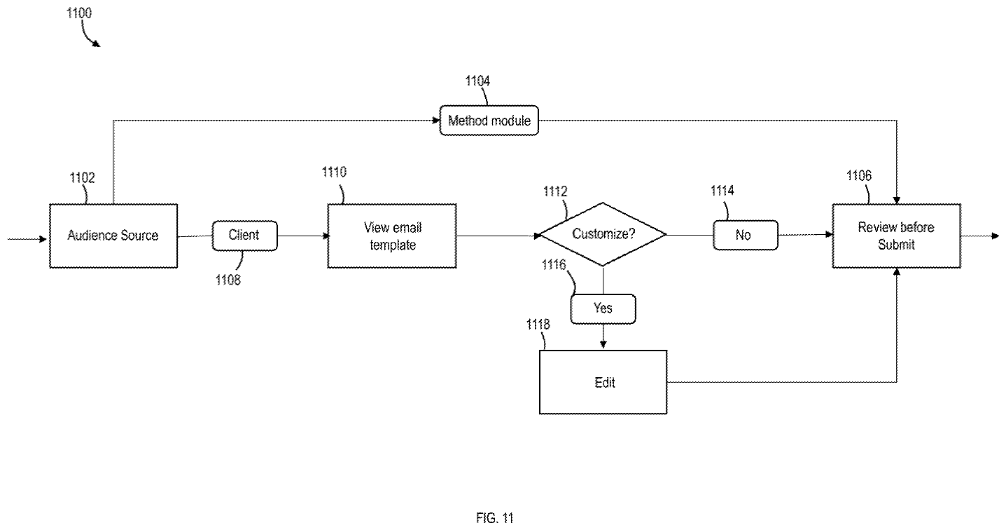

Referring now to FIG. 11, shown therein is a method flow 1100 for a deployment and user review module, according to an embodiment.

Clients want to deploy surveys to respondents via email and SMS. Clients want the ability to define delivery criteria based on profiling answers. If a survey is going out to a client's own community, the client often wants some control over the messaging. There may be multiple brand communities under a client. Each brand community may have a different look and feel (e.g., logo and brand colours) for the survey and communication. Managers want to see a preview of the survey and communication that will be sent to their community.

The deployment and user review module may allow a user to deploy a survey to survey respondents via electronic message such as email or short message service (SMS). The deployment and user review module may allow a user to define survey delivery criteria based on profiling answers. If a survey is going out to a market researcher's own community, the market research will likely want some control over the messaging. Further, there may be multiple “brand” communities (e.g., a direct banking brand, bank employees, bank customers). Each brand community may have a different look and feel (e.g., logo and brand colours) for the survey and communication. Further, market researcher users often want to see a preview of the survey and communication that will be sent to their community.

While FIG. 11 refers to email, other types of electronic messages (e.g. SMS) are contemplated.

At 1102, an audience source is determined. The audience source may be a method module 1104 (e.g. AskingCanadians, RFG, Cint, etc.) or a client's custom audience 1108. Clients may have the ability to upload their own emailing list to contact.

At 1104, the audience source is the method module (e.g. Methodify™) and emails are delivered to the correct respondents without the need for the user to take any action.

At 1108, a client is selected and proceeds to view email template module 1110.

At 1110, the view email template module is invoked and generates a user interface displaying an email template to be sent to survey respondents. The user can review the email template in the user interface. The email is used to deploy the survey and provides access to the survey (e.g. by providing a link) and message content.

At 1112, the user interface displays a user interface element allowing the user to indicate whether he wishes to customize the email template.

At 1114, input data indicating the user does not wish to customize the email template is received and the flow proceeds to the review before submit module 1106.

At 1116, input data indicating the user wishes to customize the email template is received and the flow proceeds to a template editing module at 1118.

At 1118, the template editing module generates and displays a user interface configured to allow the user to edit message content of the email template. Message content that may be edited includes, for example, a message subject, a message heading, message paragraph text, and message call-to-action text for the respondent to enter the survey.

The received input data is used by the system to generate an edited email template.

Once editing is complete, the flow proceeds to the review before submit module 1106.

The review before submit module 1106 generates and displays a user interface including the email that is to go out to respondents (edited or unedited). The user interface may be configured to send the email to survey respondents in response to receiving input data confirming the email,

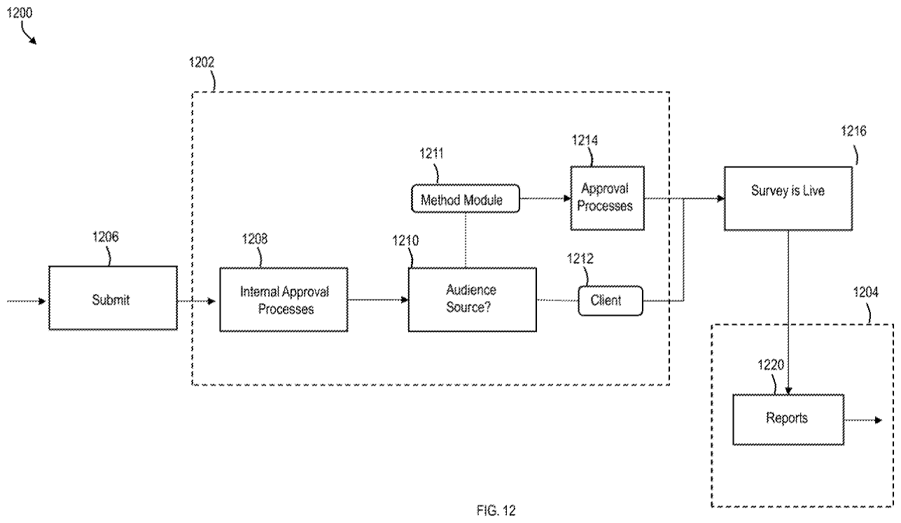

Referring now to FIG. 12, shown therein is a method flow 1200 for a going live module, according to an embodiment.

The going live module may provide live reporting data (e.g., live performance report) such rate of survey opens, click-throughs, completions, and the like while the method/project is live and collecting responses.

The going live module includes an approvals component 1202 and a reporting component 1204.

At 1206, the user may submit a method. Submission of the method by the user initiates an approval process implemented by the approvals component 1202.

At 1208, depending on the client hierarchy and governance structure, an internal client approval process may be performed as the client's team member reviews the survey content for quality issues (such as bias) or errors. The team member may also have budgeting approval to launch the project.

During the steps of 1210 to 1214, depending on the audience selected, the platform determines which process is used to set the project to Live 1216. If a method module audience is selected at 1211, the project is reviewed by method module operations (e.g. method module service provider) to validate the survey and audience is set up correctly before going Live 1216. If a client audience is selected at 1212, the client can go directly to Live status at 1216.

At 1216, the survey is deployed and goes live. Once the survey is live, response data is collected by the system via the deployed surveys.

Survey response data collected via the live survey is provided to a reporting module at 1220. The reporting module is configured to generate reporting data indicating performance of the survey (e.g., responses collected, click-throughs, completions, survey opens, etc.). The reporting data is displayed in a report. The report may be a PDF or displayed in a web-based interface.

Clients often want to understand the key insights of a report as quickly as possible, (e.g., what option performed best on a survey). Existing approaches to reporting survey results have become too complicated for ease of understanding of insights. When a method or project of the present application is live and collecting responses, clients may want to see a live performance report with data such as rate of opens, click-throughs, completions, etc. Clients may want to share reports by sending a link back to the report in situ or as a PDF.

In another aspect, there is a virtual moderator capable of asking survey respondents questions about the options and concepts selected by the respondents (i.e. based on response data provided by the respondent). The virtual moderator may be configured to ask respondents to expand upon or clarify unsatisfactory or otherwise unclear answers. The virtual moderator may be configured to analyze received response data provided by the respondent and determine that a response or answer is unsatisfactory or unclear. The virtual moderator may be further configured to generate a request that can be displayed to the respondent which requests the respondent to expand on or clarify a particular answer or response.

While the above description provides examples of one or more apparatus, methods, or systems, it will be appreciated that other apparatus, methods, or systems may be within the scope of the claims as interpreted by one of skill in the art.

1. A computer system for automated market research, the system comprising:
a research automation server platform configured to:
store data collection method template data;
generate an electronic data collection method using the data collection method template data and researcher input data;
receive response data via an input interface based on a respondent interaction with the electronic data collection method;
generate market research insight data based on the response data; and
display the market research insight data via an output interface; and
wherein the research automation server platform comprises:
a client layer software component, a services layer software component, and a technology layer software component;
wherein the client layer software component and the services layer software component communicate via a client-services application programming interface (“API”) layer; and
wherein the services layer software component and the technology layer software component communicate via a services-technology API layer.

2. The system of claim 1, wherein the client layer software component is configured to automate or virtualize a process performed by the research automation server platform.

3. The system of claim 1, wherein the response data includes a survey participant response to market research data presented via the data collection method.

4. The system of claim 3, wherein the market research data includes any one or more of an image, a video, or an interactive prototype.

5. The system of claim 1, wherein the researcher input data includes method selection data, audience selection data, and market research data.

6. The system of claim 1, wherein the research automation server platform is further configured to generate an audience of respondents for the electronic data collection method using the researcher input data.

7. The system of claim 6, wherein the audience is a virtual audience comprising a simulated model of a customer segment.

8. The system of claim 7, wherein the electronic data collection method is configured to test against the simulated model of the customer segment in real-time.

9. The system of claim 1, wherein the research automation server platform is further configured to define an audience for the electronic data collection in response to input data provided by a user, and wherein the audience is an existing audience stored by the research automation server platform and previously created by the user using the research automation server platform.

10. The system of claim 1, wherein the research automation server platform is further configured to define an audience for the electronic data collection method using audience configuration data provided by a user and store the audience configuration data such that the audience configuration data can be used to define an audience for a subsequent electronic data collection method.

11. The system of claim 1, wherein the response data is provided by a virtual participant.

12. The system of claim 11, wherein the virtual participant represents a plurality of participants.

13. The system of claim 1, wherein the research automation platform includes a method selection module for selecting the electronic data collection method from a plurality of data collection methods.

14. The system of claim 13, wherein the method selection module provides research needs data to a virtual recommendation engine in communication with the method selection module and the virtual recommendation engine generates method suggestion data including at least one of the plurality of data collection methods.

15. The system of claim 1, wherein the research automation server platform includes a virtualization module which implements a virtual researcher configured to perform any one or more of recommending a data collection method, identifying insights from the response data, and validating a concept tested using the electronic data collection method.

16. The system of claim 1, wherein the electronic data collection method includes a chatbot configured to conduct an interview with a respondent.

17. The system of claim 1, wherein the electronic data collection method collects response data using any one or more of facial coding, emotion detection, eye-tracking, biometrics, or neuro sensors.

18. The system of claim 1, wherein the market research insight data is continually updated as new response data is received.

19. The system of claim 1, wherein the response data is generated by a machine learning model configured to simulate a human response to a particular question or stimuli.

20. The system of claim 1, wherein the market research insight data is generated using emotional analysis, the emotional analysis performed using an artificial intelligence system configured to simulate a human emotional response to stimuli presented in the electronic data collection method.
